(19) United States
(12) Patent Application Publication
(10) Pub. No.: US 2021/0178105 A1
(43) Pub. Date: Jun. 17, 2021

(54) ANION FALL GENERATING DEVICE AND METHOD

(71) Applicants: Jun Gao, Wuhan City (CN); Zhihui Gong, Wuhan City (CN); Yifan Gao, Wuhan City (CN); Zhenqiang Liu, Wuhan City (CN)

(72) Inventors: Jun Gao, Wuhan City (CN); Zhihui Gong, Wuhan City (CN); Yifan Gao, Wuhan City (CN); Zhenqiang Liu, Wuhan City (CN)

(73) Assignee: Wuhan Jielang Environmental Protection Science & Technology Co., Ltd.

(21) Appl. No.: 16/714,773

(22) Filed: Dec. 15, 2019

(51) Int. Cl. A61M 16/12; A61M 16/00; A61M 16/16

(52) U.S. Cl. CPC A61M 16/12; A61M 2202/0208; A61M 16/16; A61M 16/0066

(57) ABSTRACT

An anion fall generating device and method, which comprises a water mist generator, an anion generator and an airflow propulsion unit. The water mist particles generated by the water mist generator and the anions generated by the anion generator are fused with each other under the effect of airflow propulsion unit, forming a water mist anion fall. The water mist particles are used as carriers, the water mist particles and anions are fused with each other, so that the water mist particles enwrap or adhere to the anions, preventing the anions from being neutralized by positive charges, so as to form the anion fall using water mist particles as carriers. Meanwhile the water mist particles can prolong the lifespan of anions in the air, and the anions are propagated farther by airflow propulsion unit, the anion generating efficiency is increased greatly.

### BACKGROUND OF INVENTION

### 1. Field of the Invention

The present invention relates generally to the technical field of anion generating device product and method, and more particularly to an anion fall generating device and method.

### 2. Description of Related Art

The aero-anion is also known as “air vitamin”, which can improve human lungs, promote metabolism, enhance resistance to diseases and regulate central nervous system. The negative oxygen ions of small particle size are likely to act on medical care through human blood-brain barrier. The scientific research indicates that the content of anion in the air is an important factor which determines air quality. An amount of anions in the air not only removes dust, eliminates bacteria and purifies air efficiently, but also activates the oxygen molecules in the air to form oxygen-carrying anions. The anions exist everywhere in nature, such natural phenomena as thundering and lightning, photosynthesis of plants and waterfall impact can generate a lot of anions.

As the aero-anions are beneficial to human health, the anion generator occurs, which has been used in many electric appliances. In terms of conventional anion generator, the pulse-generating circuit of anion generator boosts the low voltage to DC negative high voltage through the high voltage module, the electrode tip generates negative DC high corona continuously, a lot of electrons (e−) are emitted at a high speed, but the electrons cannot exist in the air for long, they are caught by the oxygen and nitrogen molecules in the air immediately, so as to form aero-anions. The operating principle is coincident with the anion generation by natural phenomenon “thundering and lightning”.

The anion generator can generate aero-anions, but the generated anions are likely to be neutralized by the positive charges in the air, so their lifespan is limited, and the propagation distance is limited. Therefore, the range of use of the present anion generators is limited, failing to form an anion fall. If the distance is too long, the effect of anions is very limited.

On the other hand, the humidifier is a device for atomizing moisture. According to the atomization methods, the humidifiers are classified into direct evaporation humidifier, ultrasonic high frequency oscillation humidifier and so on, the moisture is atomized to fine particles, so as to increase the air humidity.

This inventor overcomes the defects in the existing anion generators, combines humidifier with anion generator through continuous research and development, and proposes the following technical proposal.

### SUMMARY OF THE INVENTION

The first technical problem to be solved by the present invention is to overcome the shortcomings of the prior art and provide an anion fall generating device, comprising a water mist generator, an anion generator and an airflow propulsion unit; the water mist particles generated by the water mist generator and the anions generated by the anion generator are fused with each other under the action of the airflow propulsion unit to form a water mist anion fall.

More particularly, wherein the water mist generator is located in an upper end of anion generator, the water mist generation outlet of the water mist generator faces down, the water mist particles generated by the water mist generator are fused with the anions generated by the anion generator when moving downward through the water mist generation outlet to form a water mist anion fall.

More particularly, wherein the airflow propulsion unit is located in the lower end of anion generator, the anion generator is distributed on the lateral side of water mist generation outlet, the airflow generated by the airflow propulsion unit makes the water mist particles and anions collected and fused, forming a water mist anion fall.

More particularly, wherein the airflow propulsion unit is located between the water mist generator and anion generator, the anion generator is distributed on the lateral side of air flow path of airflow propulsion unit, the airflow propulsion unit pushes the water mist particles to move downward to be fused with anions, forming a water mist anion fall.

More particularly, wherein the water mist generator is located on the lateral side of anion generator, the water mist generation outlet of the water mist generator faces down, the water mist particles generated by the water mist generator are fused with the anions generated by the anion generator when moving downward through the water mist generation outlet, forming a water mist anion fall.

More particularly, the airflow propulsion unit is located in the lower end of water mist generator and anion generator, the airflow generated by the airflow propulsion unit makes the water mist particles and anions collected and fused towards the airflow propulsion unit, forming a water mist anion fall.

More particularly, wherein the water mist generator uses a high frequency ultrasonic oscillation plate to generate water mist particles.

More particularly, wherein the anion generator uses a high voltage corona device with a point electrode needle.

More particularly, wherein the airflow propulsion unit is a fan or a blower.

The second technical problem to be solved by the present invention is to overcome the shortcomings of the prior art and provide a particle waterfall generating method.

In order to solve the above technical problems, the present invention adopts the following technical solutions:

An anion fall generation method, comprising:
I. a water mist generator generating water mist particles, a anion generator generating anions;
II. a airflow propulsion unit or external air flow making the water mist particles and anions fused with each other;
III. the anions and water mist particles are fused, the water mist particles enwrap or adhere to the anions, forming an anion fall using water mist particles as carriers, so as to prolong the lifespan of anions in the air.

The technical proposal of the present invention overcomes the defects in the existing anion generators, such as insufficient aero-anions generated by the existing anion generators, short propagation distance of anions, short lifespan of anions in the air, low generating efficiency and limited effect.

The present invention uses water mist particles as carriers, the water mist particles and anions are fused with each other, so that the water mist particles enwrap or adhere to the anions, preventing the anions from being neutralized by positive charges, so as to form the anion fall using water mist particles as carriers. Meanwhile the water mist particles can prolong the lifespan of anions in the air, and the anions are propagated farther by airflow propulsion unit, the anion generating efficiency is increased greatly.

### BRIEF DESCRIPTION OF THE DRAWINGS

FIG. 1 is a schematic diagram of a first embodiment of the present invention;

FIG. 2 is a schematic diagram of a second embodiment of the present invention;

FIG. 3 is a schematic diagram of a third embodiment of the present invention;

FIG. 4 is a schematic diagram of a fourth embodiment of the present invention;

FIG. 5 is a schematic diagram of a fifth embodiment of the present invention.

### DETAILED DESCRIPTION OF THE INVENTION

The details of the present invention are described with the figures below.

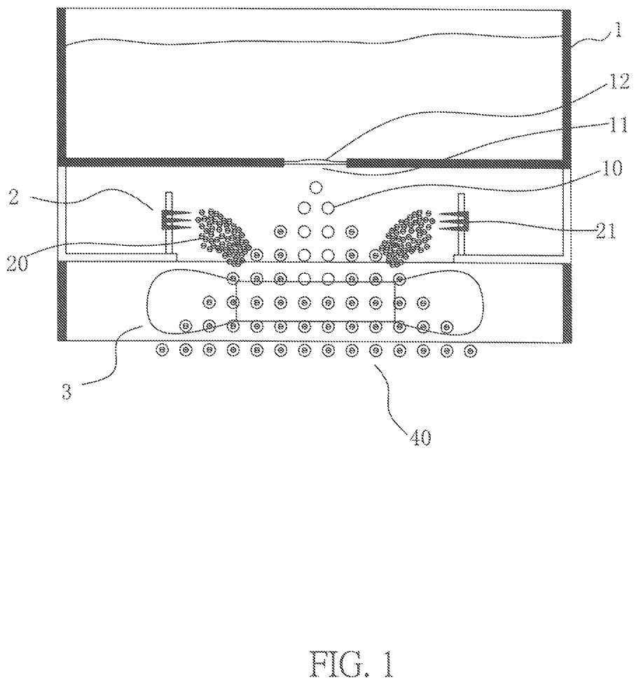

The present invention proposes an anion fall generation method, as shown in FIGS. 1 to 5, the anion fall generation method is:

First, the water mist generator 1 generates water mist particles 10, the anion generator 2 generates anions 20;

Secondly, the airflow propulsion unit 3 or external air flow makes the water mist particles 10 fused with anions 20;

Finally, the anions 20 and water mist particles 10 are fused with each other, the water mist particles 10 enwrap or adhere to the anions 20, forming an anion fall 40 using water mist particles 20 as carriers, so as to prolong the lifespan of anions 20 in the air.

The present invention proposes an anion fall generating device, as shown in FIGS. 1 to 5.

FIG. 1 shows the Embodiment 1 of the present invention, the aforementioned anion fall generating device comprises a water mist generator 1, an anion generator 2 and an airflow propulsion unit 3. The water mist particles 10 generated by the aforementioned water mist generator 1 and the anions 20 generated by the anion generator 2 are fused with each other under the effect of airflow propulsion unit 3, forming a water mist anion fall 40.

To be specific, the water mist generator 1 is located in the upper end of anion generator 2, the water mist generation outlet 11 of the water mist generator 1 faces down, the water mist particles 10 generated by water mist generator 1 are fused with the anions 20 generated by the anion generator 2 when running downward through the water mist generation outlet 11, forming a water mist anion fall 40.

Said airflow propulsion unit 3 is located in the lower end of anion generator 2. The anion generator 2 is distributed over the lateral side of the water mist generation outlet 11, the airflow generated by the airflow propulsion unit 3 makes the water mist particles 10 fused with anions 20, forming a water mist anion fall 40.

The water mist generator 1 in the present invention is high frequency ultrasonic oscillation plate water mist generator, the particle size of the generated water mist particles 10 is less than 1 μm. According to test, the smaller the particle size of water mist particles 10 is, the better is the effect. Therefore, the present invention prefers nano atomization generator.

The anion generator 2 of the present invention is a high voltage corona device with a point electrode needle 21.

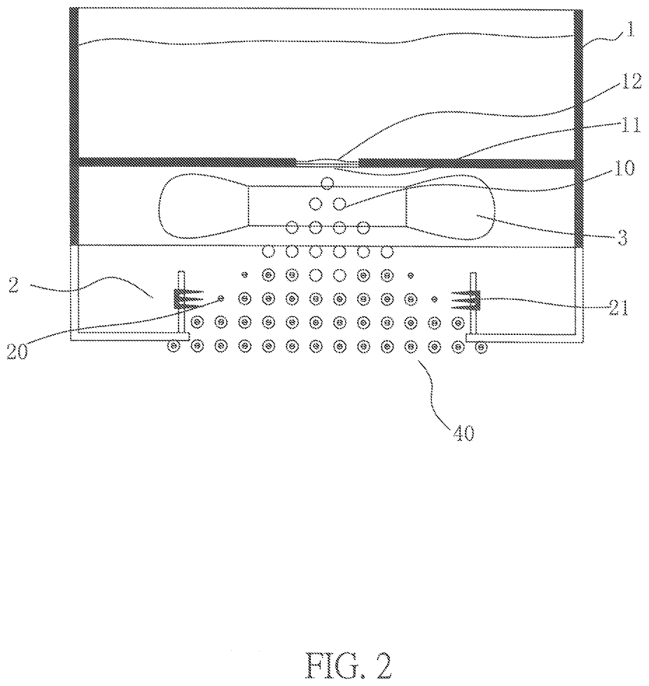

FIG. 2 shows Embodiment 2 of the present invention. The difference from Embodiment 1 is that in this embodiment, the aforementioned airflow propulsion unit 3 is located between water mist generator 1 and anion generator 2. The anion generator 2 is distributed on the lateral side of air flow path of airflow propulsion unit 3, the airflow propulsion unit 3 pushes the water mist particles 10 to move downward to be fused with anions 20, forming a water mist anion fall 40. Embodiment 2 uses the airflow propulsion unit 3 to collect the water mist particles 10, and then they are fused with anions.

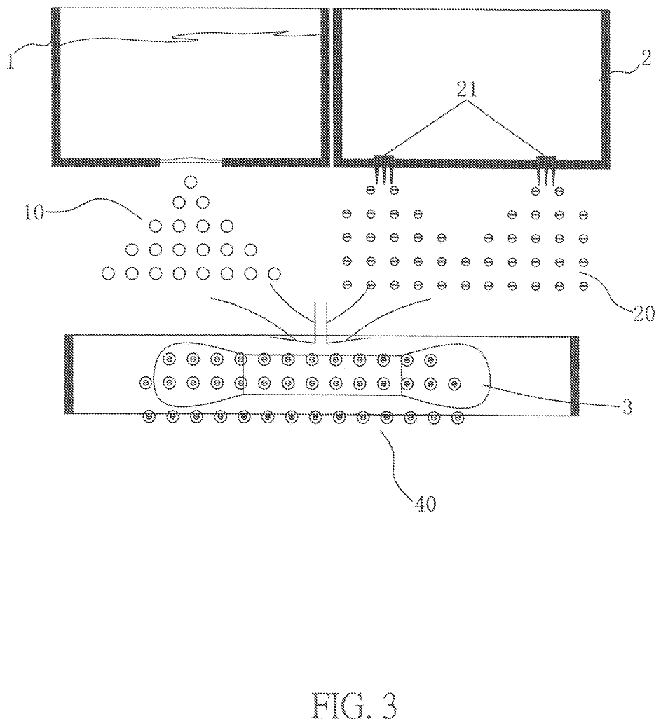

FIG. 3 shows the Embodiment 3 of the present invention, the difference from Embodiments 1 and 2 is that the aforementioned water mist generator 1 and anion generator 2 are placed in parallel in this embodiment, meaning the water mist generator 1 is located on the lateral side of anion generator. The water mist generation outlet 11 of the water mist generator 1 faces down, the water mist particles 10 generated by water mist generator 1 are fused with the anions 20 generated by the anion generator 2 when running downward through the water mist generation outlet 11, forming a water mist anion fall 40. Certainly, for better fusion of water mist particles 10 and anions 20, the aforementioned airflow propulsion unit 3 is located in the lower end of water mist generator 1 and anion generator 2, under the airflow effect generated by the airflow propulsion unit 3, the water mist particles 10 and anions 20 are collected and fused towards the airflow propulsion unit 3, forming a water mist anion fall 40.

Said embodiments have used an airflow propulsion unit 3, the airflow propulsion unit 3 contributes to the water mist particles 10, anions 20, water mist anion fall 40 and air flow, making the water mist particles 10 fused with anions 20, so that the formed water mist anion fall 40 is propagated farther. The airflow propulsion unit 3 of the present invention is a device which can propel the air, e.g. a fan, a blower, an axial fan or a centrifugal fan. For example, the airflow propulsion unit 4 in the Embodiment 1 is an axial fan.

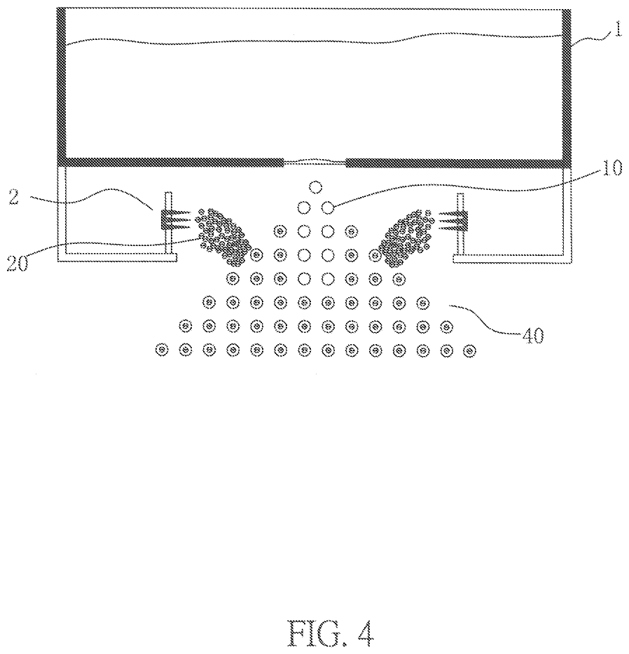

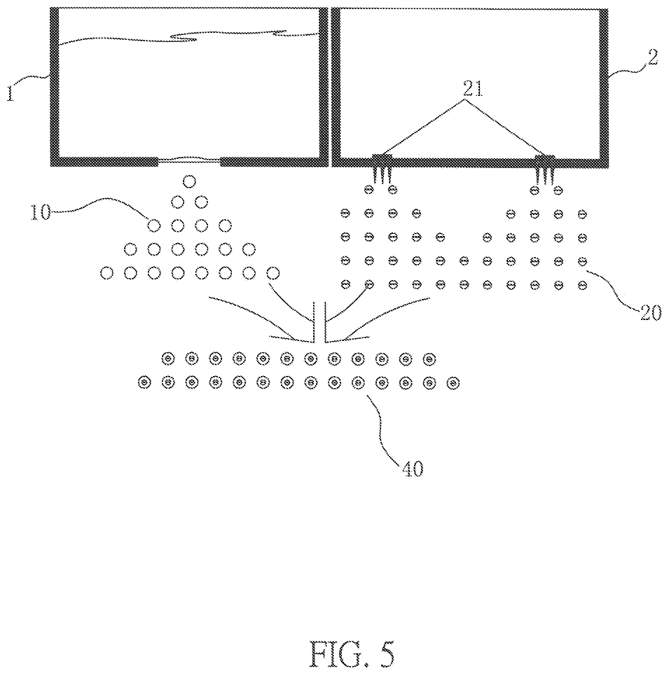

Certainly, the airflow propulsion unit 3 in the present invention can be directly combined with the water mist generator 1 and anion generator 2 to form one unit. If the present invention is integrated into other sets of equipment, which are provided with an airflow propulsion unit 3 to make the air flow, under the effect of the external airflow propulsion unit 3, the water mist particles 10 and anions 20 can be fused, forming a water mist anion fall 40, as shown in FIGS. 4 and 5.

Said embodiments of the present invention do not limit the scope of the present invention, any equivalent changes or modifications according to the aforementioned structure of the present invention shall be covered within the scope of the present invention.

We claim:

1. An anion fall generating device, comprising a water mist generator (1), an anion generator (2) and an airflow propulsion unit (3); the water mist particles (10) generated by the water mist generator (1) and the anions (20) generated by the anion generator (2) are fused with each other under the action of the airflow propulsion unit (3) to form a water mist anion fall (40).

2. The anion fall generating device defined in claim 1, wherein the water mist generator (1) is located in an upper end of anion generator (2), the water mist generation outlet (11) of the water mist generator (1) faces down, the water mist particles (10) generated by the water mist generator (1) are fused with the anions (20) generated by the anion generator (2) when moving downward through the water mist generation outlet (11) to form a water mist anion fall (40).

3. The anion fall generating device defined in claim 2, wherein the airflow propulsion unit (3) is located in the lower end of anion generator (2), the anion generator (2) is distributed on the lateral side of water mist generation outlet (11), the airflow generated by the airflow propulsion unit (3) makes the water mist particles (10) and anions (20) collected and fused, forming a water mist anion fall (40).

4. The anion fall generating device defined in claim 2, wherein the airflow propulsion unit (3) is located between the water mist generator (1) and anion generator (2), the anion generator (2) is distributed on the lateral side of air flow path of airflow propulsion unit (3), the airflow propulsion unit (3) pushes the water mist particles (10) to move downward to be fused with anions (20), forming a water mist anion fall (40).

5. The anion fall generating device defined in claim 1, wherein the water mist generator (1) is located on the lateral side of anion generator (2), the water mist generation outlet (11) of the water mist generator (1) faces down, the water mist particles (10) generated by the water mist generator (1) are fused with the anions (20) generated by the anion generator (2) when moving downward through the water mist generation outlet (11), forming a water mist anion fall (40).

6. The anion fall generating device defined in claim 5, the airflow propulsion unit (3) is located in the lower end of water mist generator (1) and anion generator (2), the airflow generated by the airflow propulsion unit (3) makes the water mist particles (10) and anions (50) collected and fused towards the airflow propulsion unit (3), forming a water mist anion fall (40).

7. The anion fall generating device defined in claim 1, wherein the water mist generator (1) uses a high frequency ultrasonic oscillation plate (12) to generate water mist particles (10).

8. The anion fall generating device defined in claim 1, wherein the anion generator (2) uses a high voltage corona device with a point electrode needle (21).

9. The anion fall generating device defined in claim 1, wherein the airflow propulsion unit (3) is a fan or a blower.

10. An anion fall generation method, comprising:
I. a water mist generator (1) generating water mist particles (10), a anion generator (2) generating anions (20);
II. a airflow propulsion unit (3) or external air flow making the water mist particles (10) and anions (20) fused with each other;
III. the anions (20) and water mist particles (10) are fused, the water mist particles (10) enwrap or adhere to the anions (20), forming an anion fall (40) using water mist particles (20) as carriers, so as to prolong the lifespan of anions (20) in the air.
